(19) United States
(12) Patent Application Publication
(10) Pub. No.: US 2023/0234054 A1
(43) Pub. Date: Jul. 27, 2023

(54) APPARATUS AND METHOD FOR SEPARATING FLUID

(71) Applicant: SPECLIPSE, INC., Gyeonggi-do (KR)

(72) Inventors: Sung Hyun PYUN, Seoul (KR); Wan Ki MIN, Seoul (KR)

(21) Appl. No.: 17/702,888

(22) Filed: Mar. 24, 2022

(30) Foreign Application Priority Data
Jan. 27, 2022 (KR) 10-2022-0012313

(51) Int. Cl. B01L 3/00

(52) U.S. Cl. CPC B01L 3/502753; B01L 2300/0816; B01L 2300/0681

(57) ABSTRACT

A fluid separation apparatus is disclosed. The fluid separation apparatus includes a diluter which includes a first filter channel through which a fluid flows, a fluid structure formed to protrude outward from the first filter channel and including an air bag, and a first vibration generator configured to generate a first sound wave and which is configured to filter at least one substance included in the fluid based on a vortex of the fluid formed on an interface between the first filter channel and the air bag, and a separator which includes a second filter channel through which the diluted fluid flows, a second vibration generator configured to generate a second sound wave passing through the second filter channel, and a plurality of outlet channels branched from the second filter channel, wherein a plurality of substances included in the diluted fluid are separated based on the molecular weight.

### CROSS-REFERENCE TO RELATED APPLICATION

This application claims priority to and the benefit of Korean Patent Application No. 2022-0012313, filed on Jan. 27, 2022, the disclosure of which is incorporated herein by reference in its entirety.

### FIELD

The present disclosure relates to an apparatus and method for separating a fluid, and more particularly, to an apparatus and method for separating a fluid capable of separating a substance contained in a fluid.

### BACKGROUND

Currently, in bioindustry, research and commercialization of point-of-care (POC) and lab-on-a-chip (LOC: which means a laboratory on a chip and indicates a technology that can diagnose various diseases at once within a small chip) are being actively carried out. Biochemical tests on biological samples such as blood are widely conducted using the POC or LOC.

Blood (whole blood) consists of blood cell components such as red blood cells, white blood cells, and platelets, and plasma components such as water, protein, fat, carbohydrates, and other mineral ions. When blood is analyzed, the blood cell components act as active components that affect the analysis results. Thus, in order to obtain accurate analysis results, it is necessary to conduct a test using only the plasma components without the blood cell components. For example, when spectral analysis of blood is performed using Raman spectroscopy, accurate analysis results may not be obtained due to the blood cell components being combined with nanoparticles for biomarker detection. Therefore, there is a need for a technology that can separate the blood cell components and plasma components from blood. This technology is referred to as blood separation technology.

Conventionally, a large centrifugal separator has been used to separate blood cells and plasma. Although it can reliably separate blood, the centrifugal separator has to be used in a limited environment such as a laboratory and thus is difficult to use it in the field.

Accordingly, there is a need for an apparatus capable of separating blood in an undiluted state.

### SUMMARY

The present disclosure is directed to providing a fluid separation apparatus that is applicable to point-of-care (POC).

The present disclosure is also directed to providing a blood separation apparatus capable of separating blood in an undiluted state.

The objectives of the present disclosure are not limited to those mentioned above, and other unmentioned objectives should be clearly understood by those of ordinary skill in the art to which the present disclosure pertains from the description below.

One exemplary embodiment of the present disclosure provides a fluid separation apparatus including: a loader which includes an inlet through which a fluid is injected and an inlet channel through which the fluid flows; a diluter which includes a first filter channel through which the fluid passing through the inlet channel flows, a fluid structure formed to protrude outward from the first filter channel and including an air bag, and a first vibration generator configured to generate a first sound wave and which is configured to filter at least one substance contained in the fluid based on a vortex of the fluid formed on an interface between the first filter channel and the air bag, wherein the vortex is generated based on the first sound wave; and a separator which includes a second filter channel through which the diluted fluid passing through the first filter channel flows, a second vibration generator configured to generate a second sound wave passing through the second filter channel, and a plurality of outlet channels branched from the second filter channel, wherein a plurality of substances included in the diluted fluid are separated according to the molecular weight based on the second sound wave and flow through the plurality of outlet channels.

One exemplary embodiment of the present disclosure provides a fluid separation method including: receiving a first fluid; obtaining a second fluid by a fluid structure, which is formed to protrude outward from a channel through which the first fluid flows and which includes an air bag, vibrating to filter at least one substance contained in the first fluid; and separating a plurality of substances contained in the second fluid according to the molecular weight by generating a sound wave encountering the second fluid flowing along the channel.

The means for achieving the objectives of the present disclosure are not limited to those described above, and other unmentioned means should be clearly understood by those of ordinary skill in the art to which the present disclosure pertains from this specification and the accompanying drawings.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view of a fluid separation apparatus from the top according to an embodiment of the present disclosure;

FIG. 2 is a view of the fluid separation apparatus from the bottom according to an embodiment of the present disclosure;

FIG. 3 is a view of the fluid separation apparatus from the bottom according to an embodiment of the present disclosure;

FIG. 4 is a view illustrating a diluter according to an embodiment of the present disclosure;

FIG. 5 is a view illustrating a separator according to an embodiment of the present disclosure;

FIG. 6 is a view illustrating the separator according to an embodiment of the present disclosure;

FIG. 7 is a view illustrating a driving signal according to a first embodiment of the present disclosure;

FIG. 8 is a view illustrating a driving signal according to a second embodiment of the present disclosure;

FIG. 9 is a view illustrating a fluid separation apparatus according to an embodiment of the present disclosure; and

FIG. 10 is a view for describing a fluid separation method according to an embodiment of the present disclosure.

### DETAILED DESCRIPTION

Because embodiments described herein are for clearly describing the spirit of the present disclosure to one of ordinary skill in the art to which the present disclosure pertains, the present disclosure is not limited by the embodiments described herein, and the scope of the present disclosure should be construed as including modifications or alterations that do not depart from the spirit of the present disclosure.

Terms used herein are selected general terms that are currently widely used in the art to which the present disclosure pertains, but the terms may vary depending on the intention or practice of one of ordinary skill in the art to which the present disclosure pertains or the advent of new technology. However, on the other hand, when a specific term is arbitrarily defined and used, a definition of the term will be separately given. Consequently, the terms used herein should be interpreted on the basis of substantial meanings thereof and all content herein instead of being interpreted simply on the basis of the names of the terms.

Terms such as first and second may be used to describe various elements, but the elements are not limited by the terms. The terms are only used for the purpose of distinguishing one element from another element.

A singular expression includes a plural expression unless the context clearly indicates otherwise. In this application, terms such as “include” or “have” should be understood as specifying that features, number, steps, operations, elements, components, or combinations thereof are present and not as precluding the possibility of the presence or addition of one or more other features, numbers, steps, operations, elements, components, or combinations thereof in advance.

The accompanying drawings are for easily describing the present disclosure, and the shapes illustrated in the drawings may be exaggerated or reduced as necessary to help the understanding of the present disclosure. Thus, the present disclosure is not limited by the drawings.

In the description, when detailed description of a known configuration or function related to the present disclosure is deemed as having the possibility of obscuring the gist of the present disclosure, the detailed description thereof may be omitted as necessary.

One exemplary embodiment of the present disclosure may provide a fluid separation apparatus including: a loader which includes an inlet through which a fluid is injected and an inlet channel through which the fluid flows; a diluter which includes a first filter channel through which the fluid passing through the inlet channel flows, a fluid structure formed to protrude outward from the first filter channel and including an air bag, and a first vibration generator configured to generate a first sound wave and which is configured to, on the basis of the first sound wave, filter at least one substance contained in the fluid on the basis of a vortex of the fluid formed on an interface between the first filter channel and the air bag; and a separator which includes a second filter channel through which the diluted fluid passing through the first filter channel flows, a second vibration generator configured to generate a second sound wave passing through the second filter channel, and a plurality of outlet channels branched from the second filter channel and through which a plurality of substances contained in the diluted fluid, which are separated according to the molecular weight on the basis of the second sound wave, flow.

The air bag may pump the fluid according to contraction and expansion based on the first sound wave.

The injected fluid may be undiluted whole blood, and the at least one substance may include blood cells.

A frequency of the first sound wave may be in a range of several kHz to several hundreds of kHz, and a frequency of the second sound wave may be in a range of several MHz to several hundreds of MHz.

The fluid separation apparatus may further include a vibration isolator configured to isolate a first vibration based on the first sound wave and a second vibration based on the second sound wave.

The first vibration generator may generate the first sound wave during a first time window, and the second vibration generator may generate the second sound wave during a second time window which does not overlap with the first time window.

The first vibration generator may generate the first sound wave during the first time window and may reduce the strength of the first sound wave in a third time window of the first time window that overlaps with the second time window during which the second vibration generator generates the second sound wave.

The plurality of substances contained in the diluted fluid may include at least one substance filtered by the diluter.

The fluid separation apparatus may further include a driver configured to apply a driving signal to the first vibration generator and the second vibration generator, and the driver may apply a first driving signal to the first vibration generator so that the first vibration generator generates the first sound wave having a first frequency and may apply a second driving signal to the second vibration generator so that the second vibration generator generates the second sound wave having a second frequency higher than the first frequency.

The driver may include a first driver configured to output the first driving signal and a second driver configured to output the second driving signal.

The second filter channel may include a region in which the plurality of substances move while forming an acute angle with a direction of the second filter channel.

The fluid structure may form an obtuse angle with a direction of the first filter channel so that the fluid is pumped by the air bag.

The second sound wave may advance in a direction perpendicular to the direction of the second filter channel.

The plurality of substances may flow so that the higher the molecular weight, the farther the substance flows from the center of the second filter channel.

The size of the at least one substance may correspond to the frequency of the first sound wave.

One exemplary embodiment of the present disclosure may provide a fluid separation method including: receiving a first fluid; obtaining a second fluid by a fluid structure, which is formed to protrude outward from a channel through which the first fluid flows and which includes an air bag, vibrating to filter at least one substance contained in the first fluid; and separating a plurality of substances contained in the second fluid according to the molecular weight by generating a sound wave encountering the second fluid flowing along the channel.

The obtaining of the second fluid may include pumping the first fluid in a direction of the channel by the air bag contracting and expanding on the basis of the vibration of the fluid structure.

The obtaining of the second fluid may include forming a vortex of the first fluid on an interface between the channel and the air bag and holding the at least one substance by the vortex of the first fluid.

The separating may include separating the plurality of substances so that the higher the molecular weight, the farther the position of the flowing substance from the center of the channel.

The fluid structure may form an obtuse angle with the direction of the channel so that the first fluid is pumped in the direction of the channel.

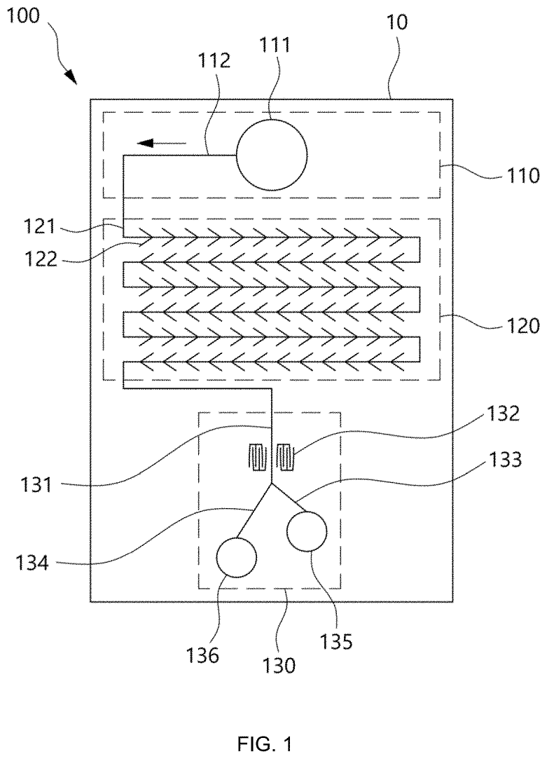

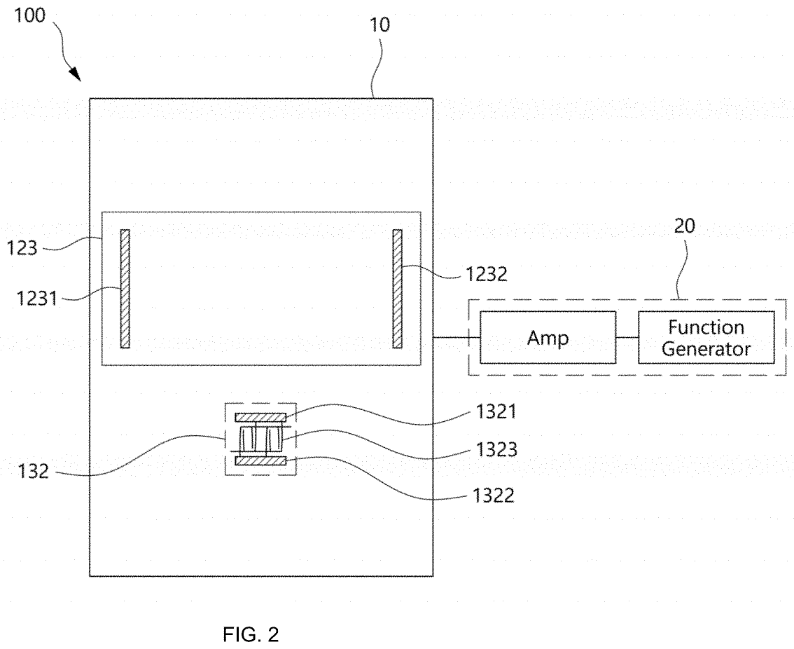

FIG. 1 is a view of a fluid separation apparatus from the top according to an embodiment of the present disclosure. FIG. 2 is a view of the fluid separation apparatus from the bottom according to an embodiment of the present disclosure.

Referring to FIG. 1, a fluid separation apparatus 100 may include a substrate 10, a loader 110, a diluter 120, and a separator 130.

The loader 110 may receive a fluid and transport the received fluid to the diluter 120. The loader 110 may include an inlet 111 through which a fluid is injected and an inlet channel 112 through which the fluid flows. The fluid may be undiluted whole blood. The loader 110 may be disposed above the substrate 10.

The diluter 120 is a configuration for filtering at least one substance contained in the fluid. The diluter 120 may deliver the fluid with a reduced concentration of the at least one substance to the separator 130. For example, the at least one substance may include at least one of red blood cells, white blood cells, and platelets.

The diluter 120 may include a first filter channel 121 through which the fluid passing through the inlet channel 112 flows, a fluid structure 122 formed to protrude outward from the first filter channel 121, and a first vibration generator 123. The first filter channel 121 may be disposed above the substrate 10. The first vibration generator 123 may be disposed below the substrate 10. For example, the fluid structure 122 and the first vibration generator 123 may constitute a lateral cavity acoustic transducer (LCAT).

The fluid structure 122 may include an air bag that the fluid does not enter. An interface may be formed between the air bag and the first filter channel 121. When the first vibration generator 123 generates a first sound wave, vibration due to the first sound wave may be delivered to the first filter channel 121 and the fluid structure 122. As the first filter channel 121 and the fluid structure 122 vibrate, a vortex of the fluid may be formed in a region in the first filter channel 121 that is adjacent to the interface between the air bag and the first filter channel 121. At least some of the blood cell components contained in the fluid flowing through the first filter channel 121 may be trapped in the vortex of the fluid.

When the vibration of the first filter channel 121 and the fluid structure 122 stops, the vortex of the fluid may disappear. Here, at least some of the blood cell components trapped in the vortex of the fluid may move again along the first filter channel 121.

The frequency of the first sound wave may correspond to the size of the substance trapped in the vortex of the fluid. For example, the frequency of the first sound wave may correspond to the size of red blood cells or the size of white blood cells.

The air bag included in the fluid structure 122 may repeat contraction and expansion on the basis of the first sound wave generated by the first vibration generator 123. Accordingly, the air bag may pump the fluid in a direction of the first filter channel 121. That is, the air bag may perform a pumping function that causes the fluid introduced into the first filter channel 121 to move to the separator 130. The direction of the first filter channel 121 may be the same as a direction in which the fluid flows in the first filter channel 121.

The fluid structure 122 may be formed to protrude outward from the first filter channel 121. A direction in which the fluid structure 122 protrudes may form an obtuse angle with the direction of the first filter channel 121. Accordingly, the pumping effect in which the air bag causes the fluid to move may be increased. That is, since the pumping effect increases with higher similarity between the direction of the channel and a direction in which the fluid is pushed, the fluid structure 122 of which the protruding direction forms an obtuse angle with the direction of the first filter channel 121 may be a structure advantageous for causing the fluid to move.

The first vibration generator 123 may include a piezoelectric transducer. The first vibration generator 123 may generate the first sound wave on the basis of a driving signal input thereto. The frequency of the first sound wave is in a range of several kHz to several hundreds of kHz.

Meanwhile, the number of blood cell components filtered by the diluter 120 may be increased with a longer length of the first filter channel 121. However, when the length of the first filter channel 121 is reduced to manufacture the fluid separation apparatus 100 in a small size, blood cell components not filtered by the diluter 120 may be present. Therefore, there is a need to provide a configuration for secondarily filtering the blood cell components not filtered by the diluter 120.

The separator 130 is a configuration for separating a plurality of substances contained in the fluid according to the molecular weight. The separator 130 may include a second filter channel 131, a second vibration generator 132, a plurality of outlet channels 133 and 134, and a plurality of chambers 135 and 136. The second filter channel 131 may be disposed above the substrate 10. The second vibration generator 132 may be disposed below the substrate 10. However, this is only an exemplary embodiment, and the second vibration generator 132 may also be disposed above the substrate 10.

The second filter channel 131 may receive the fluid discharged from the diluter 120. The second filter channel 131 may receive the diluted fluid passing through the first filter channel 121. A concentration of the at least one substance (e.g., red blood cells or white blood cells) contained in the diluted fluid may be lower than a concentration of the at least one substance (e.g., red blood cells or white blood cells) contained in the fluid injected through the inlet 111.

The second vibration generator 132 may generate the second sound wave. The second sound wave may advance across a direction of the second filter channel 131. The second sound wave may advance in a direction perpendicular to the direction of the second filter channel 131. The second sound wave may be a surface acoustic wave (SAW). The frequency of the second sound wave may be in a range of several MHz to several hundreds of MHz.

The second vibration generator 132 may include an electrode pattern. The electrode pattern may include an inter-digital transducer (IDT) electrode.

When the second filter channel 131 vibrates due to the second sound wave, a moving direction of the plurality of substances contained in the diluted fluid may be changed. When the second sound wave encounters the diluted fluid, the moving direction of the plurality of substances contained in the diluted fluid may be changed. The moving direction of the plurality of substances may be changed on the basis of the molecular weight. For example, a first substance and a second substance moving along a first line of the second filter channel 131 may be present. The molecular weight of the first substance (e.g., blood cell components) may be higher than the molecular weight of the second substance (e.g., plasma components). When the second filter channel 131 vibrates, the moving direction of the first substance may be changed so that the first substance moves at a first position spaced a first distance from the first line of the second filter channel 131. When the second filter channel 131 vibrates, the moving direction of the second substance may be changed so that the second substance moves at a second position a second distance, which is smaller than the first distance, from a central region of the second filter channel 131.

The outlet channels 133 and 134 may receive the fluid containing the substances whose moving direction is changed on the basis of the second sound wave. For example, a first outlet channel 133 may receive a first fluid not containing the first substance. The first fluid may contain the second substance. A second outlet channel 134 may receive a second fluid containing the first substance.

The fluids flowing through the outlet channels 133 and 134 may be stored in the chambers 135 and 136. For example, a first chamber 135 may store the first fluid. A second chamber 136 may store the second fluid.

In this way, the separator 130 may separate blood cell components not filtered by the diluter 120 from plasma components to secondarily filter the blood cell components. Meanwhile, attempts have been made to separate blood using a SAW, but there has been a problem in that the separation performance is degraded when the number of blood cell components input exceeds a threshold value. A method of adding diluted blood to a blood separator has been used to overcome this problem, but this method has an inconvenience that a separate task of diluting blood should be performed.

The blood separation apparatus 100 according to the present disclosure primarily filters blood cell components using the diluter 120, thus having an effect of diluting blood. Since blood input to the separator 130 is blood diluted by the diluter 120, it is possible to accurately separate blood even when the SAW is used. Therefore, the blood separation apparatus 100 has an advantage of being able to separate undiluted blood.

Various analysis processes may be performed on the fluid separated through the separator 130. For example, an analysis process may be performed on the first fluid stored in the first chamber 135. The analysis process may include antigen-antibody testing, fluorescence testing, DNA/RNA testing, and laser testing. The laser testing may include Raman analysis, surface-enhanced Raman spectroscopy, and absorption analysis.

Meanwhile, the channels defined in the present disclosure may be connected to each other by a fluid transfer structure and form a single flow path. In the present disclosure, a case where the fluid is blood is mainly described for convenience of description, but the fluid may be any of various other liquids collected from a test subject. For example, the fluid may be urine, saliva, semen, sweat, tears, or cerebrospinal fluid.

Referring to FIG. 2, the first vibration generator 123 and the second vibration generator 132 may be disposed below the substrate 10. The first vibration generator 123 may include a first electrode 1231 disposed in a first direction from the center of the first vibration generator 123 and a second electrode 1232 disposed in a second direction from the center of the first vibration generator 123.

The second vibration generator 132 may be an IDT electrode. The IDT electrode may include a plurality of bars 1321 and 1322 facing each other and a plurality of fingers 1323 protruding from the plurality of bars 1321 and 1322.

According to an embodiment, the first vibration generator 123 and the second vibration generator 132 may receive a driving signal from an external source 20 and generate a sound wave on the basis of the received driving signal. The external source 20 may include a function generator and an amplifier. The driving signal may be an electrical signal corresponding to a sound wave to be generated. The fluid separation apparatus 100 may include an interface (e.g., a connector, a port, or the like) for connection to the external source 20.

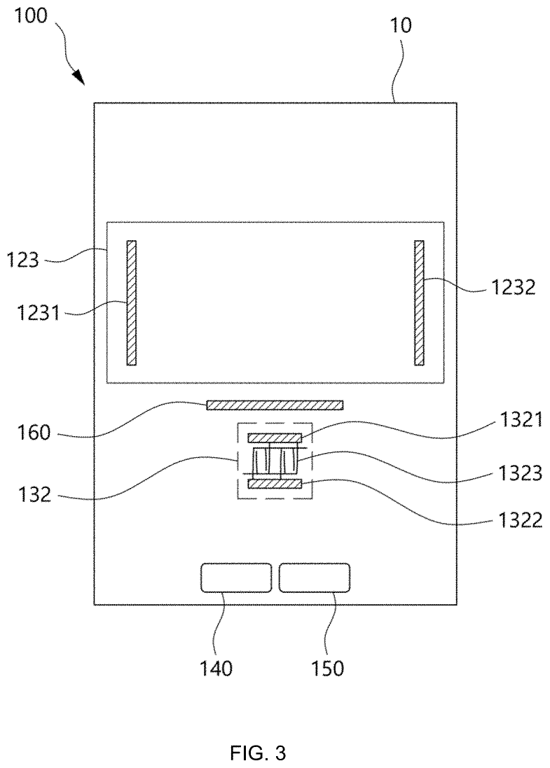

FIG. 3 is a view of the fluid separation apparatus from the bottom according to an embodiment of the present disclosure.

Referring to FIG. 3, the fluid separation apparatus 100 may include a driver 140 configured to apply a driving signal to the first vibration generator 123 and the second vibration generator 132, a battery 150 configured to provide power to the driver 140, and a vibration isolator 160 configured to isolate a first vibration generated by the first vibration generator 123 and a second vibration generated by the second vibration generator 132.

The vibration isolator 160 may be disposed between the first vibration generator 123 and the second vibration generator 132. The vibration isolator 160 may absorb a portion of the first vibration generated by the first vibration generator 123. Alternatively, the vibration isolator 160 may absorb a portion of the second vibration generated by the second vibration generator 132. Accordingly, the first vibration and the second vibration may be isolated from each other. The vibration isolator 160 may include an elastic member (e.g., rubber).

The driver 140 may be electrically connected to the first vibration generator 123 and the second vibration generator 132. The driver 140 may be disposed below the substrate 10.

The driver 140 may apply different driving signals to the first vibration generator 123 and the second vibration generator 132. For example, the driver 140 may apply a first driving signal to the first vibration generator 123 so that the first vibration generator 123 generates the first sound wave having a first frequency. The driver 140 may apply a second driving signal to the second vibration generator 132 so that the second vibration generator 132 generates the second sound wave having a second frequency.

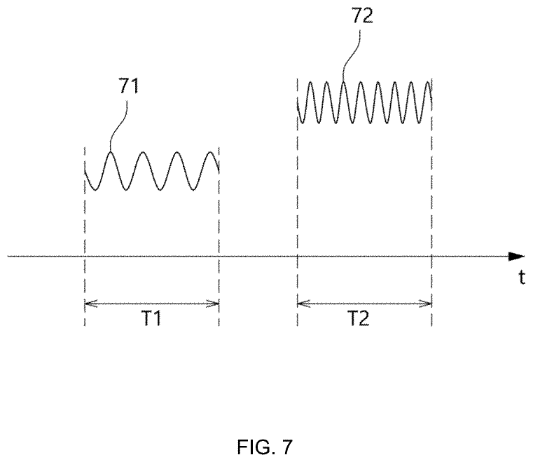

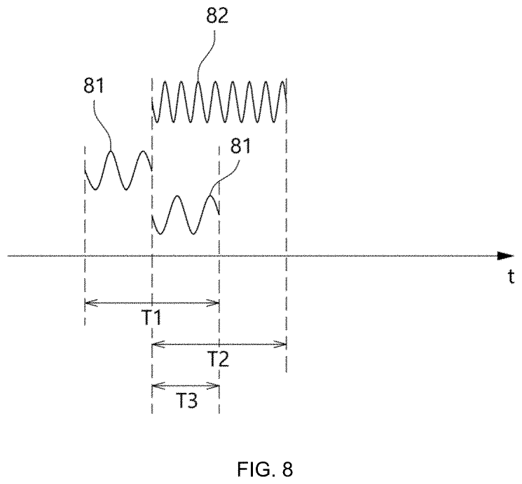

The driver 140 may include a first driver configured to apply the first driving signal and a second driver configured to apply the second driving signal. Alternatively, the driver 140 may output the first driving signal and the second driving signal together, and a frequency division apparatus (e.g., a frequency filter) may divide the first driving signal and the second driving signal from each other and transmit the first driving signal and the second driving signal to the first vibration generator 123 and the second vibration generator 132. The driver 140 will be described in more detail below with reference to FIGS. 7 and 8.

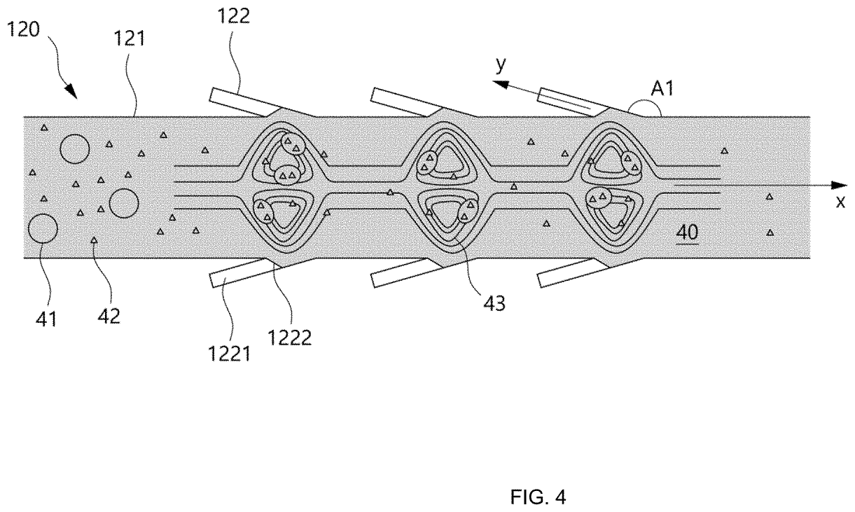

FIG. 4 is a view illustrating the diluter according to an embodiment of the present disclosure.

Referring to FIG. 4, the diluter 120 may include the first filter channel 121 and the fluid structure 122 formed to protrude outward from the first filter channel 121. The fluid structure 122 may form an obtuse angle with a direction x of the first filter channel 121. That is, an angle A1 between the direction x of the first filter channel 121 and a protruding direction y of the fluid structure 122 may be in a range of 90° to 180°.

A first fluid 40 flowing through the first filter channel 121 may include a first substance 41 and a second substance 42. The first substance 41 may be one of the blood cell components, and the second substance 42 may be one of the plasma components.

The fluid structure 122 may include an air bag 1221 that the first fluid 40 does not enter. The air bag 1221 may serve as a pump. The air bag 1221 may repeat contraction and expansion on the basis of the first sound wave generated by the first vibration generator 123. Accordingly, the air bag 1221 may pump the first fluid 40 in the direction x of the first filter channel 121.

The fluid structure 122 may serve as a filter. When the first sound wave having a frequency corresponding to the size of the first substance 41 is generated from the first vibration generator 123, a vortex 43 of the first fluid 40 may be formed in a region near an interface 1222 between the first filter channel 121 and the air bag 1221. The first substance 41 may be trapped in the vortex 43 of the first fluid 40. Accordingly, the fluid structure 122 may filter the first substance 41.

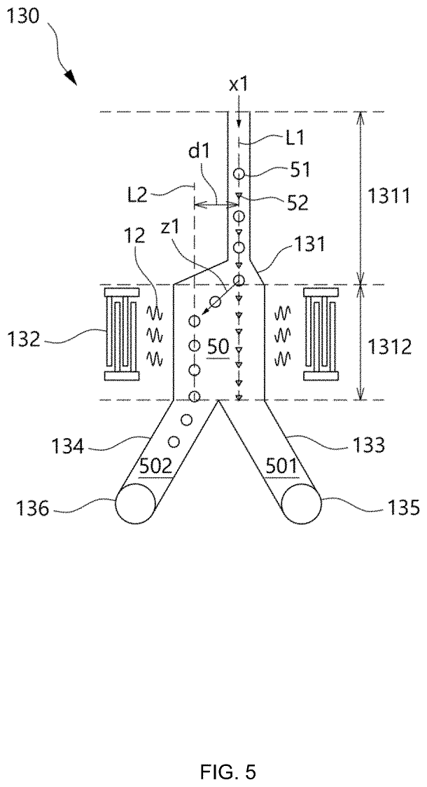

FIG. 5 is a view illustrating the separator according to an embodiment of the present disclosure.

Referring to FIG. 5, the separator 130 may include the second filter channel 131, the second vibration generator 132, the first outlet channel 133, the second outlet channel 134, the first chamber 135, and the second chamber 136.

The second filter channel 131 may include a first region 1311 and a second region 1312. The first region 1311 may be a region on which the second sound wave generated by the second vibration generator 132 does not act, and the second region 1312 may be a region on which the second sound wave acts. The second sound wave may advance across the second filter channel 131 in a direction perpendicular to a direction x1 of the second filter channel 131.

The second filter channel 131 may receive a second fluid 50. The second fluid 50 may be a fluid diluted by the diluter 120. Therefore, a concentration of the blood cell components in the second fluid 50 may be lower than a concentration of the blood cell components in the first fluid 40. The second fluid 50 may contain a first substance 51 and a second substance 52. A molecular weight of the first substance 51 may be higher than a molecular weight of the second substance 52. For example, the first substance 51 may be red blood cells, and the second substance 52 may be protein.

The first substance 51 and the second substance 52 may move along a first line L1 in the first region 1311. In a partial region of the second region 1312, a moving direction of the first substance 51 may be changed due to the second sound wave. For example, the moving direction of the first substance 51 may be changed so that the first substance 51 moves along a second line L2 spaced a first distance dl from the first line L1. Here, a moving direction z1 of the first substance 51 may form an acute angle with the direction x1 of the second filter channel 131. After the moving direction is changed, the first substance 51 may move along the second line L2.

The degree of change in the direction in the second region 1312 may be higher with a higher molecular weight of the substance. For example, the degree of change in the direction of the first substance 51, whose molecular weight is higher than the molecular weight of the second substance 52, may be higher than the degree of change in the direction of the second substance 52. In this way, due to the second sound wave, the plurality of substances contained in the second fluid 50 may be separated according to the molecular weight.

The first substance 51 passing through the second filter channel 131 may enter the second outlet channel 134. In the first chamber 135, a fluid 501 not containing the first substance 51 may be obtained. The second substance 52 passing through the second filter channel 131 may enter the first outlet channel 133. In the second chamber 136, a fluid 502 not containing the second substance 52 may be obtained.

Meanwhile, the separator 130 may have an asymmetrical structure as illustrated in FIG. 5, or may have a symmetrical structure. Also, the number of outlet channels may be three or more.

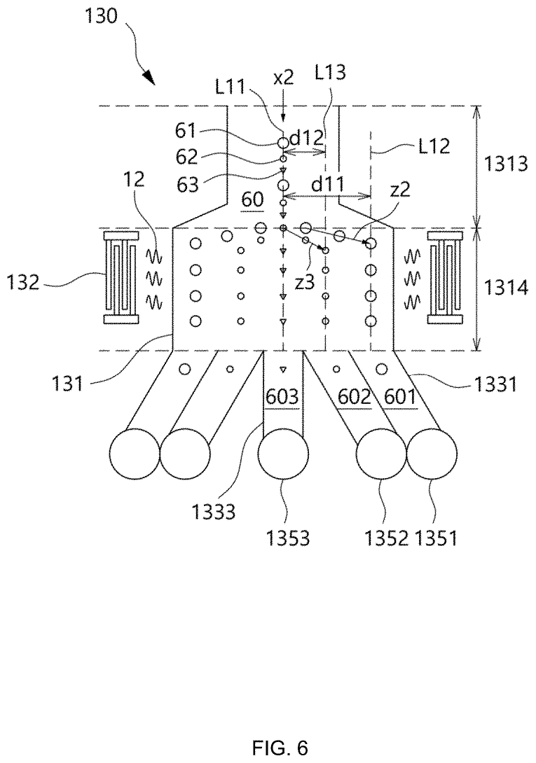

FIG. 6 is a view illustrating the separator according to an embodiment of the present disclosure.

Referring to FIG. 6, the separator 130 may include the second filter channel 131, the second vibration generator 132, the first outlet channel 133, the second outlet channel 134, the first chamber 135, and the second chamber 136.

The second filter channel 131 may include a first region 1313 and a second region 1314. The first region 1313 may be a region on which the second sound wave generated by the second vibration generator 132 does not act, and the second region 1314 may be a region on which the second sound wave acts.

The second filter channel 131 may receive a third fluid 60 containing a first substance 61, a second substance 62, and a third substance 63. A molecular weight of the first substance 61 may be higher than a molecular weight of the second substance 62, and the molecular weight of the second substance 62 may be higher than a molecular weight of the third substance 63. For example, the first substance 61 may be white blood cells, the second substance 62 may be red blood cells, and the third substance 63 may be protein.

The first substance 61, the second substance 62, and the third substance 63 may move along a first line L11 in the first region 1313. In a partial region of the second region 1314, a moving direction of the first substance 61 and the second substance 62 may be changed. For example, the moving direction of the first substance 61 may be changed so that the first substance 61 moves along a second line L12 spaced a first distance d11 from the first line L11. The moving direction of the second substance 62 may be changed so that the second substance 62 moves along a third line L13 spaced a second distance d12 from the first line L11.

A moving direction z2 of the first substance 61 and a moving direction z3 of the second substance 62 may form an acute angle with a direction x2 of the second filter channel 131. A first angle formed between the moving direction z2 of the first substance 61 and the direction x2 of the second filter channel 131 may be larger than a second angle formed between the moving direction z3 of the second substance 62 and the direction x2 of the second filter channel 131.

The plurality of substances 61, 62, and 63 passing through the second filter channel 131 may enter a plurality of outlet channels 1331, 1332, and 1333, respectively. A plurality of fluids 601, 602, and 603 containing the plurality of substances 61, 62, and 63, respectively, may be accommodated in a plurality of chambers 1351, 1352, and 1353.

Meanwhile, in a case where the first sound wave and the second sound wave having different frequencies are simultaneously generated, interference may occur between a first vibration based on the first sound wave and a second vibration based on the second sound wave. In order to prevent this, it is necessary to control a driving signal applied to each vibration generator.

FIG. 7 is a view illustrating a driving signal according to a first embodiment of the present disclosure.

Referring to FIG. 7, the driver 140 may apply a first driving signal 71 to the first vibration generator 123 of the diluter 120 and apply a second driving signal 72 to the second vibration generator 132 of the separator 130. Accordingly, the first vibration generator 123 may generate the first sound wave, and the second vibration generator 132 may generate the second sound wave.

A frequency of the first driving signal 71 may be lower than a frequency of the second driving signal 72. For example, the frequency of the first driving signal 71 may be in a range of several kHz to several hundreds of kHz, and the frequency of the second driving signal 72 may be in a range of several MHz to several hundreds of MHz. The size of the first driving signal 71 may be smaller than the size of the second driving signal 72. However, this is only an exemplary embodiment, and the size of the first driving signal 71 may also be larger than the size of the second driving signal 72.

In order to prevent the interference between the first vibration based on the first sound wave and the second vibration based on the second sound wave, the driver 140 may output the first driving signal 71 and the second driving signal 72 at different timings. For example, the driver 140 may output the first driving signal 71 during a first time window T1. The driver 140 may output the second driving signal 72 during a second time window T2. Accordingly, the first vibration generator 123 may generate the first sound wave during the first time window T1. The second vibration generator 132 may generate the second sound wave during the second time window T2.

A buffer window may be present between the first time window T1 and the second time window T2. A length of the buffer window may be calculated on the basis of a physical distance between the first filter channel 121 and the second filter channel 131.

The length of the buffer window may be set on the basis of a point in time at which the diluted fluid discharged from the diluter 120 is expected to enter the separator 130. That is, the driver 140 may output the second driving signal 72 on the basis of a point in time at which the diluted fluid is expected to enter the separator 130. To this end, the fluid separation apparatus 100 may include a fluid detection sensor. The fluid detection sensor may be provided between the diluter 120 and the separator 130.

In this way, by controlling the driving signal applied to each of the first vibration generator 123 and the second vibration generator 132, the interference between the first vibration generated by the first vibration generator 123 and the second vibration generated by the second vibration generator 132 may be prevented.

FIG. 8 is a view illustrating a driving signal according to a second embodiment of the present disclosure.

Referring to FIG. 8, the driver 140 may apply a first driving signal 81 to the first vibration generator 123 and apply a second driving signal 82 to the second vibration generator 132. Meanwhile, a gap between the diluter 120 and the separator 130 may be minimized to manufacture the fluid separation apparatus 100 in a small size. Accordingly, a third time window T3 in which a first time window T1, during which the first driving signal 81 is output, and a second time window T2, during which the second driving signal 82 is output, overlap with each other may be present.

The strength of the first driving signal 81 may change over time in the first time window T1. For example, the first driving signal 81 may have a first strength in a window of the first time window T1 that does not overlap with the second time window T2. The first driving signal 81 may have a second strength, which is lower than the first strength, in the third time window T3. The second strength may be 0. That is, while the second vibration generator 132 generates the second sound wave, the first vibration generator 123 may reduce the strength of the first sound wave or stop generating the first sound wave.

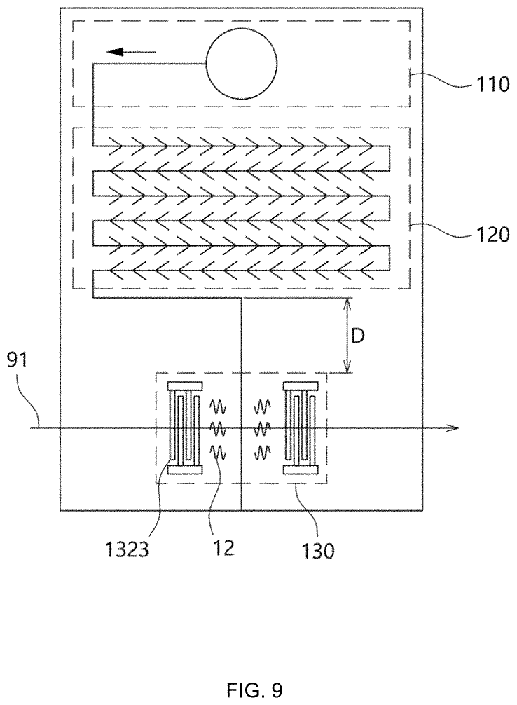

FIG. 9 is a view illustrating the fluid separation apparatus according to an embodiment of the present disclosure.

Referring to FIG. 9, the diluter 120 and the separator 130 may be disposed a first distance D from each other. The first distance D may be larger than a predetermined distance (e.g., 2 cm). Accordingly, the interference between the first vibration generated in the diluter 120 and the second vibration generated in the separator 130 may be reduced or prevented.

A first axis 91 which is perpendicular to the direction in which the plurality of fingers 1323 included in the separator 130 protrude may be defined. An axis extending in the direction in which the plurality of fingers 1323 protrude may be defined as a second axis. That is, the second axis may be perpendicular to the first axis 91.

A second sound wave 12 generated by the separator 130 may advance in a direction of the first axis 91. In order to reduce the interference between the first vibration and the second vibration, the diluter 120 may be disposed at a position where the advancing of the second sound wave 12 does not occur. For example, the diluter 120 may be disposed to be placed on the second axis with respect to the separator 130. The direction in which the plurality of fingers 1323 protrude may be horizontal to a virtual line extending along the center of the diluter 120 and the center of the separator 130.

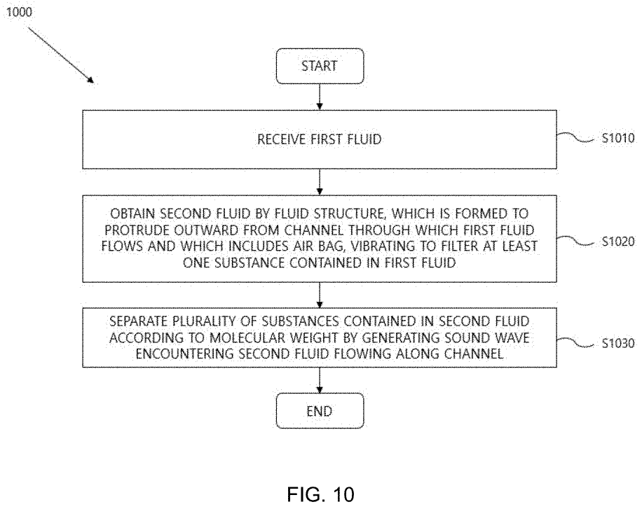

FIG. 10 is a view for describing a fluid separation method according to an embodiment of the present disclosure.

Referring to FIG. 10, a fluid separation method 1000 may include receiving a first fluid (S1010). The first fluid may contain undiluted whole blood.

The fluid separation method 1000 may include obtaining a second fluid by a fluid structure, which is formed to protrude outward from a channel through which the first fluid flows and which includes an air bag, vibrating to filter at least one substance contained in the first fluid (S1020). The obtaining of the second fluid (S1020) may be performed using the diluter 120.

The obtaining of the second fluid (S1020) may include pumping the first fluid in a direction of the channel by the air bag contracting and expanding on the basis of the vibration of the fluid structure. The fluid structure may form an obtuse angle with the direction of the channel so that the first fluid is pumped in the direction of the channel.

The obtaining of the second fluid (S1020) may include forming a vortex of the first fluid on an interface between the channel and the air bag and holding the at least one substance by the vortex of the first fluid.

The fluid separation method 1000 may include separating a plurality of substances contained in the second fluid according to the molecular weight by generating a sound wave encountering the second fluid flowing along the channel (S1030). The separating (S1030) may be performed using the separator 130. The separating (S1030) may include separating the plurality of substances so that the higher the molecular weight, the farther the position of the flowing substance from the center of the channel.

According to various embodiments of the present disclosure described above, a fluid separation apparatus can separate a fluid in an undiluted state. Accordingly, the fluid separation apparatus can be applied to POC. Also, due to not requiring a pump for fluid transfer, the fluid separation apparatus can be manufactured in a small size.

Other effects obtainable or predictable from the embodiments of the present disclosure have been disclosed directly or implicitly in the detailed description of the embodiments of the present disclosure. For example, various effects predictable according to the embodiments of the present disclosure have been disclosed in the detailed description given above.

Other aspects, advantages, and salient features of the present disclosure should be apparent to those of ordinary skill in the art from the detailed description above which discloses various embodiments of the present disclosure with reference to the accompanying drawings.

The configurations and features of the present disclosure have been described above with reference to the embodiments according to the present disclosure, but the present disclosure is not limited thereto. It should be apparent to those of ordinary skill in the art to which the present disclosure pertains that various modifications or alterations are possible within the spirit and scope of the present disclosure. Thus, it should be noted that such modifications or alterations also belong to the scope of the attached claims.

What is claimed is:

1. A fluid separation apparatus comprising:
a loader which includes an inlet through which a fluid is injected and an inlet channel through which the fluid flows;
a diluter which includes a first filter channel through which the fluid passing through the inlet channel flows, a fluid structure formed to protrude outward from the first filter channel and including an air bag, and a first vibration generator configured to generate a first sound wave and which is configured to filter at least one substance contained in the fluid based on a vortex of the fluid formed on an interface between the first filter channel and the air bag, wherein the vortex is generated based on the first sound wave; and
a separator which includes a second filter channel through which the diluted fluid passing through the first filter channel flows, a second vibration generator configured to generate a second sound wave passing through the second filter channel, and a plurality of outlet channels branched from the second filter channel, wherein a plurality of substances included in the diluted fluid are separated according to the molecular weight based on the second sound wave and flow through the plurality of outlet channels.

2. The fluid separation apparatus of claim 1, wherein the air bag pumps the fluid according to contraction and expansion based on the first sound wave.

3. The fluid separation apparatus of claim 1, wherein:
the injected fluid is undiluted whole blood; and
the at least one substance includes blood cells

4. The fluid separation apparatus of claim 1, wherein:
a frequency of the first sound wave is in a range of several kHz to several hundreds of kHz; and
a frequency of the second sound wave is in a range of several MHz to several hundreds of MHz

5. The fluid separation apparatus of claim 1, further comprising a vibration isolator configured to isolate a first vibration based on the first sound wave and a second vibration based on the second sound wave.

6. The fluid separation apparatus of claim 1, wherein:
the first vibration generator generates the first sound wave during a first time window; and
the second vibration generator generates the second sound wave during a second time window which does not overlap with the first time window.

7. The fluid separation apparatus of claim 1, wherein the first vibration generator generate the first sound wave during a first time window and reduces the strength of the first sound wave in a third time window of the first time window that overlaps with the second time window during which the second vibration generator generates the second sound wave.

8. The fluid separation apparatus of claim 1, wherein the plurality of substances included in the diluted fluid include at least one substance filtered by the diluter.

9. The fluid separation apparatus of claim 1, further comprising a driver configured to apply a driving signal to the first vibration generator and the second vibration generator,
wherein the driver applies a first driving signal to the first vibration generator so that the first vibration generator generates the first sound wave having a first frequency and applies a second driving signal to the second vibration generator so that the second vibration generator generates the second sound wave having a second frequency higher than the first frequency.

10. The fluid separation apparatus of claim 9, wherein the driver includes:
a first driver configured to output the first driving signal; and
a second driver configured to output the second driving signal.

11. The fluid separation apparatus of claim 1, wherein the second filter channel includes a region in which the plurality of substances move while forming an acute angle with a direction of the second filter channel.

12. The fluid separation apparatus of claim 1, wherein the fluid structure forms an obtuse angle with a direction of the first filter channel so that the fluid is pumped by the air bag.

13. The fluid separation apparatus of claim 1, wherein the second sound wave advances in a direction perpendicular to a direction of the second filter channel.

14. The fluid separation apparatus of claim 1, wherein the plurality of substances flow so that the higher the molecular weight, the farther the substance flows from the center of the second filter channel.

15. The fluid separation apparatus of claim 1, wherein the size of the at least one substance corresponds to the frequency of the first sound wave.

16. A fluid separation method comprising:
receiving a first fluid;
obtaining a second fluid by a fluid structure, which is formed to protrude outward from a channel through which the first fluid flows and which includes an air bag, vibrating to filter at least one substance contained in the first fluid; and
separating a plurality of substances contained in the second fluid according to the molecular weight by generating a sound wave encountering the second fluid flowing along the channel.

17. The fluid separation method of claim 16, wherein the obtaining of the second fluid includes pumping the first fluid in a direction of the channel by the air bag contracting and expanding on the basis of the vibration of the fluid structure.

18. The fluid separation method of claim 16, wherein the obtaining of the second fluid includes:
forming a vortex of the first fluid on an interface between the channel and the air bag; and
holding the at least one substance by the vortex of the first fluid.

19. The fluid separation method of claim 16, wherein the separating includes separating the plurality of substances so that the higher the molecular weight, the farther the position of the flowing substance from the center of the channel.

20. The fluid separation method of claim 16, wherein the fluid structure forms an obtuse angle with the direction of the channel so that the first fluid is pumped in the direction of the channel.
